SEQUENCE LISTING (5 sequences)

SEQ ID NO: 1 - RNA, 1518 nt, Mus musculus (mouse)
gcccguugccaggcgccgccuuauaaaccucccgcucggccgccgccgcgccgaguccgagccgcgcacgggaccgggacgcagcggagcccgcgggccccgcguucaugcaccgccugcuggccugggacgcagcaugccucccgccgccgcccgccgccuuuagacccauggaaguggccaacuucuacuacgagcccgacugccuggccuacggggccaaggcggcccgcgccgcgccgcgcgcccccgccgccgagccggccauuggcgagcacgagcgcgccaucgacuucagccccuaccuggagccgcucgcgcccgccgcggacuucgccgcgcccgcgcccgcgcaccacgacuuccucuccgaccucuucgccgacgacuacggcgccaagccgagcaagaagccggccgacuacgguuacgugagccucggccgcgcgggcgccaaggccgcgccgcccgccugcuucccgccgccgccucccgccgcgcucaaggcggagccgggcuucgaacccgcggacugcaagcgcgcggacgacgcgcccgccauggcggccgguuucccguucgcccugcgcgccuaccugggcuaccaggcgacgccgagcggcagcagcggcagccuguccacgucgucgucguccagcccgcccggcacgccgagccccgccgacgccaaggccgcgcccgccgccugcuucgcggggccgccggccgcgcccgccaaggccaaggccaagaagacgguggacaagcugagcgacgaguacaagaugcggcgcgagcgcaacaacaucgcggugcgcaagagccgcgacaaggccaagaugcgcaaccuggagacgcagcacaaggugcuggagcugacggcggagaacgagcggcugcagaagaagguggagcagcugucgcgagagcucagcacccugcggaacuuguucaagcagcugcccgagccgcugcuggccucggcgggccacugcuagcgcggcgcggguggcguggggggcgccgcggccacccugggcaccgugcgcccugccccgcgcgcuccggccccgcgcgcgcgcccggaccaccgugcgugcccugcgcgcaccugcaccugcaccgaggggacaccgcgggcacaccgcgggcaccgcgcgcacgcaccugcacagcgcaccggguuucgggacuugaugcaauccggaucaaacguggcugagcgcguguggacacgggacugacgcaacacacguguaacugucagccgggcccugaguaaucacuuaaagauguuccugcgggguuguugauguuuuugguuuuguuuuuguuuuuuguuuuguuuuguuuuuuuuuuuggucuuauuauuuuuuuuguauuauauaaaaaaguucuauuucuaugagaaaagaggcguauguauauuuugagaaccuuuuccguuucgagcauuaaagugaagacauuuuaauaaacuuuuuuggagaauguuuaaaagccuuuugggggcagua

SEQ ID NO: 2 - RNA, 2651 nt, Mus musculus (mouse)
auucgcgacccgaagcugcgcgggcgcgagccaguuggggcacugggugggcggcggcgacagcggcgccacgcgcaggcuggaggccgccgaggcucgccaugccgggagaacucuaacucccccauggagucggccgacuucuacgagguggagccgcggcccccgaugagcagucaccuccagagccccccgcacgcgcccagcaacgccgccuuuggcuuuccccggggcgcgggccccgcgccgcccccagccccaccugccgccccggagccgcugggcggcaucugcgagcacgagacgucuauagacaucagcgccuacaucgacccggccgccuucaacgacgaguuccuggccgaccucuuccagcacagccgacagcaggagaaggccaaggcggcggcgggccccgcggguggcggcggugacuuugacuacccgggagccccggcgggccccggcggcgcggucauguccgcgggggcgcacgggcccccucccggcuacggcugugcggcggccggcuaccuggacggcaggcuggagccccuguacgagcgcgucggggcgcccgcgcuacggccgcuggugaucaaacaagagccccgcgaggaggacgaggcgaagcagcuggcgcuggccggccucuuccccuaccagccaccgccgccaccgccaccgccgcacccgcacgcgucucccgcgcaccuggccgccccccacuugcaguuccagaucgcgcacugcggccagaccaccaugcaccugcagccuggccaccccacaccgccgcccacgcccgugcccagcccgcacgcugcgcccgccuugggugcugcgggccugccuggccccgggagcgcgcucaagggcuuggccggugcgcaccccgaccuccgcacgggaggcggcggcgguggcagcggugccggugcgggcaaagccaagaagucgguggacaagaacagcaacgaguaccggguacggcgggaacgcaacaacaucgcggugcgcaagagccgagauaaagccaaacaacgcaacguggagacgcaacagaaggugcuggaguugaccagugacaaugaccgccugcgcaagcggguggaacagcugagccgugaacuggacacgcugcggggcaucuuccgccagcugccugagagcuccuuggucaaggccaugggcaacugcgcgugaggcgcgcggcugcgggaccgccuugggccggcccccuggcuggagacccagaggaugguuucgggucgcuggaucucuaggcugcccgggccgcgcaagccaggacuaggagauuccgguguggccugaaagccuggccugcuccgcguguccccucccuuccucugagccggacucggugcgucuaagaugagggagucaggccguggugguuucuccuugagaccgagagacuuuccgcggagcugagcugggggcccggcaguacuaguauuaaggaaguaaccuugugccuuggauacucaaaacucgcuccuuuuccuaccgaguagggggagcaaaaaugugccuugauauuuuauuuggaggauuccugcuuccucucgggccucagcuggccccgugagaaaaaugaagggugcaggcccagggcaggaggaagauacaggaagcugagaucccggcagugcccugagcugccccucagucccugucuuuagaggggagggacuuagguguuggggauuugagucuguguccucacccccagcuacagggagguggagggcuccuaaucccuugcuuuuugcaccuccaccuacauccccccccccccacucagcuuacaacaggccagguuuccugggugaguucauggagaaugggggcaccacccccagucagaccagaaagcugaguugugaguuagccaugugguaggagacagagaccuagguuucugggcuuuguggggugggggauaggaggacacggggaccauuagccuuguguguacuguaugucgccagccgcuguugcugaaggaacuugaagcacaaucgauccaucccagagggacuggaguuaugacaagcuucccaaauauuuugcuuuaucauccgauaucaacacuuguaucuggucucugugucccagcggugccuugugcaauggcagugugcacgucuaugcuaaaccaccauuuuauuuggucuuuuguuuuguuuugguuuugcucugauucuugccaaacugagacucuucacuaacggcugggggaaggagcugagugaggcucucauucuuuuugguuuagggauguuuggguuuuuucgucugccucccagaggaccaaugaaaugaagugggcuucccccucuccccuaguuguccaaggguguauguaguagugggucuuagcuuccuccggcuaagacuuaggcuuccccacccacccaaccccauccccaacggcccuggcucugggucuggaaagaaggccaccuccagccaguucauacacacaccccuguggcugggagcagggcuggaccgcuuccuucucuucuuuuuuuugggggggggggacacaaaguuucaugcuagaugucguauguauuauaucuauaauauaaacauaucaaacucaaaaaaaaaaaaaaaaa

SEQ ID NO: 3 - RNA, 1857 nt, Mus musculus (mouse)
gugugacgacaaggugaccgggcugaggggacgggcugaggagaagucacacucugacaggagccugugagaccaacagccugacggggucucgguugaggggacgcgggcugagaagucacguucugacaggacugugugacagacaagauuugaaagaagcggugaaccacugauauucaggacauuuuuaaaaacaagacuacccuuuacugaaauuaccaugguugacacagagaugccauucuggcccaccaacuucggaaucagcucuguggaccucuccgugauggaagaccacucgcauuccuuugacaucaagcccuuuaccacaguugauuucuccagcauuucugcuccacacuaugaagacauuccauucacaagagcugacccaaugguugcugauuacaaauaugaccugaagcuccaagaauaccaaagugcgaucaaaguagaaccugcaucuccaccuuauuauucugaaaagacccagcucuacaacaggccucaugaagaaccuucuaacucccucauggccauugagugccgagucuguggggauaaagcaucaggcuuccacuauggaguucaugcuugugaaggaugcaaggguuuuuuccgaagaaccauccgauugaagcuuauuuaugauaggugugaucuuaacugccggauccacaaaaaaaguagaaauaaaugucaguacugucgguuucagaagugccuugcuguggggaugucucacaaugccaucagguuugggcggaugccacaggccgagaaggagaagcuguuggcggagaucuccagugauaucgaccagcugaacccagagucugcugaucugcgagcccuggcaaagcauuuguaugacucauacauaaaguccuucccgcugaccaaagccaaggcgagggcgaucuugacaggaaagacaacggacaaaucaccauuugucaucuacgacaugaauuccuuaaugaugggagaagauaaaaucaaguucaaacauaucaccccccugcaggagcagagcaaagagguggccauccgaauuuuucaagggugccaguuucgauccguagaagccgugcaagagaucacagaguaugccaaaaauaucccugguuucauuaaccuugauuugaaugaccaagugacucugcucaaguaugguguccaugagaucaucuacacgaugcuggccucccugaugaauaaagauggaguccucaucucagagggccaaggauucaugaccagggaguuccucaaaagccugcggaagcccuuuggugacuuuauggagccuaaguuugaguuugcugugaaguucaaugcacuggaauuagaugacagugacuuggcuauauuuauagcugucauuauucucaguggagaccgcccaggcuugcugaacgugaagcccaucgaggacauccaagacaaccugcugcaggcccuggaacugcagcucaagcugaaucacccagaguccucucagcuguucgccaaggugcuccagaagaugacagaccucaggcagaucgucacagagcacgugcagcuacugcaugugaucaagaagacagagacagacaugagccuucacccccugcuccaggagaucuacaaggacuuguauuagcaggaaagucccacccgcugacaacguguuccuucuauugauugcacuauuauuuugagggaaaaaaaucugacaccuaagaaauuuacugugaaaaagcauuuaaaaacaaaaaguuuuagaacaugaucuauuuuaugcauauuguuuauaaagauacauuuacaauuuacuuuuaauauuaaaaauuaccacauuauaaaauu

SEQ ID NO: 4 - RNA, 646 nt, Mus musculus (mouse)
auagcacccuccugugcugcagccuuucucaccuggaagacagcuccuccucgaagguuuacaaaaugugugaugccuuugugggaaccuggaagcuugucuccagugaaaacuucgaugauuacaugaaagaagugggagugggcuuugccacaaggaaaguggcaggcauggccaagcccaacaugaucaucagcguaaauggggauuuggucaccauccggucagagaguacuuuuaaaaacaccgagauuuccuucaaacugggcguggaauucgaugaaaucaccgcagacgacaggaaggugaagagcaucauaacccuagauggcggggcccuggugcaggugcagaagugggauggaaagucgaccacaauaaagagaaaacgagauggugacaagcuggugguggaauguguuaugaaaggcgugacuuccacaagaguuuaugaaagggcaugagccaaaggaagaggccuggauggaaauuugcaucaaacacuacaauagucagucggauuuauuguuuuuuuuuaaagauaugauuuuccacuaauaagcaagcaauuaauuuuuucugaagaugcauuuuauuggauaugguuauguugauuaaauaaaaccuuuuuagacuuagaaaaaaa

SEQ ID NO: 5 - RNA, 9938 nt, Mus musculus (mouse)
guggaaucgcagcgacacggaccuguccccccgcguggcccugguguccuccucagugcagaguuuccagugugaccaagcacgcccgacccacacugcgcgcgcacagugcacaccuggcaccggccgcgagggggugggggugggaggacagagaugagggcgucgggaugagccccgcguggcccgcgcgaggccgggggcggggacggaagcaggcgggggcugcgcguuccuugugcuccagcgcgcgcccgugcagggucccggcugggggcggcgcgcgcgggcaucaccccaccggcggcggcgcgccgggucccggggcgcagccccgacgcucauuggccugggcggcgcagccaagcugucagcccauguggcguggccgcgcggggauggccgcgguuuaaauagcgccggcgcggccuagagggagccagagagacggcagcggcgucccguccaguucgccugccgcgcuccucgcuugucgucugccuccagagcccagacagagaagagccauggaggagguggugauagccgguaugucggggaaguugcccgagucagagaaccuacaggaguucugggccaaccucauuggugguguggacauggucacagaugaugacaggagauggaaggcugggcucuauggauuacccaagcggucuggaaagcugaaggaucucuccaaguucgacgccuccuuuuuugggguccaccccaagcaggcacacacaauggacccccagcuucggcugcuguuggaagucagcuaugaagcaauuguggauggagguaucaacccagccucacuccgaggaacgaacacuggcgucugggugggugugagugguucagaggcauccgaggcccuuagcagagaucccgagacgcuucugggcuacagcauggugggcugccagcgugcaaugauggccaaccggcucucuuucuucuucgacuucaaaggaccaagcauugcccuggacacagccugcuccuccagcuugcuggcacuacagaaugccuaccaggccauccguaguggggaaugccccgcggcccuugugggugggaucaaccugcuccugaagccgaacaccucugugcaguucaugaagcugggcaugcucagcccggacggcaccugcagauccuuugaugauucagggaguggauauugucgcucugaggcuguuguagcaguucugcugacuaagaagucccuggcucggcgggucuaugccacgauucugaaugccggcaccaauacagauggcagcaaggagcaagguguaacauuccccucuggagaaguccaagaacaacucaucugcucucuguaucagccagcuggucuggccccggagucgcuugaguauauugaagcccauggcacgggcaccaaggugggugacccccaggaacugaauggcauuacucggucccugugcgccuuccgccaggccccucuguuaauuggcuccaccaaauccaacaugggacacccugagccugccucugggcuugcagcccugaccaaggugcuguuaucccuggagcauggggucugggccccuaaccugcacuuccacaaccccaacccugagaucccagcacuucuugaugggcggcugcagguggucgauaggccccugccuguucgugguggcaacgugggcaucaacucauuuggcuucggaggcuccaauguucaugucauccuccagcccaacacacggcaggccccugcgcccacugcacacgcugcccuuccccauuugcugcacgccaguggacgcaccuuagaggcagugcaggaccugcuggaacagggccgccagcacagccaggaccuggccuuugugagcaugcucaaugacauugcggcaaccccuacagcagccaugcccuucagggguuacacugugcuagguguugagggccguguccaagaagugcagcaaguguccaccaacaagcgcccacucugguucaucugcucagggaugggcacgcaguggcgcgggauggggcugagccucaugcgccuggacagcuuccgugagucuauccugcgcuccgaugaggcugugaagccguugggagugaaagugucagaucugcuguugagcacagaugagcgcaccuuugaugacaucgugcaugccuuugugagccucacugccauccagauugcccucaucgaccuacugacuucugugggacugaaaccugacggcaucauugggcacuccuugggagagguugccuguggcuaugcagauggcugucucucccagagagaggcugugcuugcagcuuacuggcgaggccagugcaucaaagaugcccaccucccgccuggauccauggcagcuguugguuuguccugggaggaauguaaacagcgcugccccgcuggcguggugccugccugccacaacucugaggacaccgugaccaucucuggaccucaggcugcagugaaugaauuuguggagcagcuaaagcaagaagguguguuugccaaggagguacgaacaggaggccuggcuuuccacuccuacuucauggaaggaauugcccccacauugcugcaggcucucaagaaggugauccgggaaccacggccgcgcucggcucgauggcucagcaccucuaucccugaggcccaguggcagagcagccuggcccgcacaucuucugccgaguacaaugucaacaaccuggugagcccugugcucuuccaggaagcacuguggcacaucccugagcaugccguggugcuggagauugcgccccacgcacuguugcaggcuguccugaagcgaggcgugaaguccagcugcaccaucauucccuugaugaagagggaucauaaagauaacuuggaguucuuucucaccaaccuuggcaaggugcaccucacaggcaucaaugucaacccuaacgccuuguucccaccuguggaguucccggcuccccgagggacuccucucaucuccccucacaucaagugggaccacagucagacuugggaugucccgguugcugaggacuucccaaacggcuccagcuccuccucugcuacagucuacagcaucgacgccaguccugagucgcccgaccacuaccugguagaccacugcauugacggccgggucaucuucccuggcacuggcuaccugugccugguguggaagacacuggcucgcagccugggcuugucccuagaagagaccccugugguauuugagaaugugucguuucaucaggccacuauacuacccaagacaggaaccguggcgcuggaggugaggcugcuagaggccucccaugccuuugaggugucugacacuggcaaucugauugugagcggaaaaguguaccugugggaagacccgaacuccaaguuauucgaccacccagaagucccaacacccccugagucugcaucggucucccgccugacccagggagaaguauacaaggagcugcggcugcguggcuaugauuauggcccucaguuccagggcaucugugaggccacccuugaaggugaacaaggcaagcugcucuggaaagauaacugggugaccuucauggacacaaugcugcagguauccauucuggguucuagccagcagagucuacagcuaccuacccgugugaccgccaucuauaucgacccugccacccaccgucagaagguguacaggcugaaggaggacacucaaguggcugaugugacaacgagccgcugucugggcauaacggucucuggugguauccacaucucaagacuacagacgacagcaaccucacggcggcagcaagaacagcugguccccaccuuggaaaaguucguuuucacaccgcacauggaggcugagugccugucugagagcacugcccugcagaaggagcugcaacugugcaagggucuggcacgggcucugcagaccaaggccacccagcaagggcugaaggcggcaaugcuugggcaagaggacccuccacagcacgggcugccucgacuccuggcagcugcuugccaguugcagcucaacgggaaccugcagcuggagcugggagaagcgcuggcucaagagaggcuccugcugccagaagacccucugaucaguggccuccucaacucccaggcccucaaggccugcguagacacagcccuggagaacuugucuacucucaagaugaagguggcagaggugcuggcuggagaaggccacuuguauucccgaaucccggcacugcucaacacccagcccaugcuacaacuggaauacacagccaccgaccggcacccccaggcccugaaggauguucagaccaaacugcagcagcaugauguggcgcagggccaguggaacccuuccgacccugcgcccagcagccugggugcccuugaccuucuggugugcaacugugcauuagccacccugggggauccagccuuggcccuggacaacaugguagcugcccucaaggaaggugguuuccugcuagugcacacagugcucaaaggacaugcccuuggggagacccuggccugccuacccucugaggugcagccugcgcccagccuccuaagccaggaggagugggagagccuguucucgaggaaggcacuacaccuggugggccuuaaaagguccuucuacgguacugcgcuguuccugugccggcgagccaucccacaggagaaaccuaucuuccugucuguggaggauaccagcuuccaguggguggacucucugaagagcacucuggccacguccuccucccagccuguguggcuaacggccauggacugccccaccucgggugugguggguuuggugaauugucuccgaaaagagccggguggacaccggauucgguguauccugcuguccaaccucagcaacacaucucacgcccccaaguuggacccuggcucuccagagcuacagcaggugcuaaagcaugaccucgugaugaacguguaccgggacggggccuggggugccuuccgucacuuccaguuagagcaggacaagcccaaggagcagacagcgcaugccuuuguaaacguccucacccgaggggaccucgccuccauccgcugggucuccuccccccugaagcacacgcagcccucgagcucaggagcacagcucugcacugucuacuacgccucacugaacuuccgagacaucaugcuggccacgggcaagcuguccccugaugccauuccagguaaaugggccagccgagacugcaugcucggcauggaguucucaggccgggauagguguggccggcgugugauggggcugguuccugcagaaggccuggccaccucaguccugcuaucaucugacuuccucugggauguacccuccagcuggacccuggaggaggcggccucugugcccgucgucuauaccacugcuuacuacucguuagugguucgcgggcgcauccagcguggggagaccgugcucauccacucagguucagguggugugggccaagcggccauuuccauugcccucagucugggcugccgcgucuucaccacugugggcucugcagagaagcgagcauaccuccaggccagguucccucagcuugaugacaccagcuuugccaacucgagggacacaucauuugagcagcacguguuacugcacacagguggcaaaggggucgaccugguccucaacucacuggcagaagagaagcugcaggccagugugcggugcuuggcucagcauggucgcuucuuagagauuggcaaauuugaucuuucuaacaaccacccucugggcauggcuaucuucuugaagaacgucacuuuccaugggauccugcuggacgcccuuuuugaggaggccaaugacagcuggcgggagguggcggcacuccugaaggcuggcauucgugauggagucgugaagccccucaagugcacaguguuucccaaggcccagguggaagaugccuuccgcuacauggcucaggggaaacacauuggcaaaguccuuguccagguacgggaggaggagccugaggcugugcugccaggggcucagcccacccugauuucugccaucuccaagaccuucugcccagcccauaagaguuacaucaucacugguggccuagguggcuuuggccuggagcuggcccgguggcucgugcuucgcggagcccagaggcuugugcugacuucccgaucuggaauccgcaccggcuaccaagccaagcacauucgggaguggagacgccagggcauccaagugcucgugucaacaagcaacgugagcucacuggagggggcccgugcucucaucgccgaagccacaaagcuggggcccguugggggugucuucaaccuggccaugguuuugagggaugccaugcuggagaaccagaccccagagcucuuccaggaugucaacaagcccaaauacaauggcacccugaaccuugacagggcaacccgggaagccugcccugagcuggacuacuuuguggccuucuccucuguaagcugcgggcgugguaaugcuggccaaacuaacuacggcuucgccaacucuaccauggagcguauaugugaacagcgcaggcacgauggccucccaggccuugccgugcaguggggugccauuggugacgugggcauuguccuggaagcgaugggcaccaaugacacagucaucggagguacgcugccucagcgcaucuccuccugcauggagguacuggaccucuuccugaaucagccccacgcaguccugagcagcuuugugcuggcagagaagaaagcuguggcccauggggacggggacacccagagggaucuggugaaagcuguagcacacauccuaggcauccgagaccucgcagguauuaaccuggacagcacgcuggcagaccucggccuggacucgcucauggguguggaaguucgucagauccuggaacgagaacacgaucuggugcugcccaugcgugaggugcggcagcucacgcugcggaaacuucaggaaauguccuccaagacugacucggcuacugacacgacagcccccaaguccaggagugacacgucucugaagcagaaccaacugaaccugagcacacugcuggugaacccugaggguccuacccuaacccagcucaacucggugcagagcucugagcggccucuguuccuugugcaccccauugaggguuccaccaccguguuccacagucuggcugccaagcucagugugcccaccuacggccugcagugcacccaagcugccccccuggauagcauuccgaaccuggcugccuacuacauagauugcaucaagcaagugcagccugagggacccuaccgcauagcuggguacucauuuggagccuguguagccuucgagaugugcucccagcugcaggcccagcagggcccagccccgacccacaacaaccucuuccuguuugacggcucacacaccuacguguuggccuacacccagagcuaccgggcaaagaugaccccaggcugugaagccgaggccgaggcugaggccuuaugcuucuucauaaagcaguuucuugauguggaacacagcaaggugcuggaggcccugcugccacugaagagccuggaagaucggguggcugccuccguggaccuuaucacuaagagucaccacagccuggaccgccgagagcugagcuuugcugccguguccuucuaccacaagcuccgggcagcugaucaguauaagcccaaggccaaguaccauggcaacgugacacugcugcgugccaagacaggcggcaccuauggcgaggacuugggugcugacuacaaccucucccaggugugugacgggaaggugucugugcacaucauugagggugaccaccgcacacugcuggagggcaguggccuggaauccaucaucaacaucauccauagcucccuggcugagccacgagugaguguacgggagggcuagaccugccgaccaccaugaagccacgcuccacaccugccaccagagaugcuccgauccccaccacacccugagugcaggaacuggggaggguccugcuggugggaccccuccccccaguggcccagcaccacccgcuccccugguggcugcuacaaacagaccaucacgcguguguuucccagccgcguagugggguucccagagccacugacuuggagacacccuggucugugaagagucaguggaggcaggagccaaacugagccuuuucuaccguguggcauuugccacgcuggucguuucuccauuaaauucucauauuuauugcauugcugggaaagacccccaggggugacucauuccagaacccccuaaaaugggagaagccauguggggaagauuucugggaaaguuucuagacucaauacacaggcugcuggcuggagccccuuuuugucuuguccugucccugcucacugcagggcaggauauggagagggcugguucccagggaacaaggaccccagcagacacuguagcccguggcccuugguccccagcauccccggcugccccaugaugcagggccauccugacucugcggaccgcaccgggcacugacugucuguuuuccaagacgaaaaugaugcuuggguuuugacuuuucugcagcugucagugugaagaagugucuggacugugucauuuuuacaccaaccugguaaaaaugcugcucuugaugcucuccugaucccacaauuaaacugcacgugagcgagcacugccuucgguucagucucuuuccaguccaggcccggcuucaggcucugauacuuauggguauuccccacaaguggccuccacagcucuuccagugagacuccuuggagggugugccauucugucaguauaaggggaguaugcuaaaauguguuucagcauggagggcuggcuggaggccuugccacuguaguacaagcagaauuuguccaccuuuaaguugcccugagggcaucagcuuagucacagccccgccccagcgguagagaauagcaucauccucuacuuguagucaacugaguguuggugcaagcccucuagagaaaaacguaagcaguaugugaccacugugagaagcaugucccuggaaacaggugacaugacgagagcagagggccugagucacacagaacugaccauguguguaagcagaggaguggagguugcuuggaagaggaggaucaagagaaaaguaggcucaguuuaguggacccacaguagcagccaauguaauuacauuggcuuggagcuaaggcaugguggguccugcaggguggcuuguuggacuaggcaaaguccccuggcuugaggguccaggagagacugucacaaggccgcagcauucgaucacauccagagaggagcauccagaggacaacugcauugguaggguacaaagguguucagaggaucccagucugagugauuagccuaagacugaagcaucugguuggcucuagucacacuuagaaaaauucuuacagcacucgucaugaguuguccauuccuggaaaggaccugcccaaucucuauagugucucuaccuaguuagaguuuacugacucaagggagcagauuuauaaacuugcagaaaggcccgacucccuuacgaggcugugugguccuuagaauucauggaugagaggcaauaaaaccuugagguuccuuccuguucuuugugauugccuaacaagacauaaacugggugugaugagauugcucaguggauagagcgcugugcagccugaguuugaucacuggaacccaugcagcaguagaaaagacuaaguuguccucugaccuccacacaucuguugaguggaacaagacugaaau

(19) United States
(12) Patent Application Publication
(10) Pub. No.: US 2015/0157673 A1
(43) Pub. Date: Jun. 11, 2015

(54) METHOD FOR CONTROLLING OBESITY USING Antrodia camphorata

(71) Applicant: National Taiwan Normal University, Taipei City (TW)

(72) Inventors: Po-Jung TSAI, Taipei City (TW); Chih-Ling Wang, Taipei City (TW); Cheng-Jen Chou, Taipei City (TW); Wen-Cheng Huang, Taipei City (TW)

(73) Assignee: National Taiwan Normal University, Taipei City (TW)

(21) Appl. No.: 14/188,146

(22) Filed: Feb. 24, 2014

(30) Foreign Application Priority Data
Dec. 5, 2013 (TW) 102144672

(51) Int. Cl. A61K 36/07

(52) U.S. Cl. CPC A61K 36/07

(57) ABSTRACT

A method for controlling obesity by using Antrodia camphorata is disclosed. A composition of Antrodia camphorata prepared from dried fruiting bodies of Antrodia camphorata is used to reduce lipid accumulation in adipocytes for controlling obesity, and the method includes steps of: providing a composition of Antrodia camphorata prepared from dried fruiting bodies of Antrodia camphorata; and applying the composition to reduce lipid accumulation for improving symptoms of obesity.

### CROSS REFERENCE TO RELATED APPLICATION

This application claims priority to Taiwan Application Serial Number 102144672, filed on Dec. 5, 2013, which are herein incorporated by reference.

### FIELD OF THE INVENTION

The present invention relates to a use of Antrodia camphorata, in particular to a use of Antrodia camphorata for controlling obesity and a method of using Antrodia camphorata for controlling obesity.

### BACKGROUND OF THE INVENTION

In recent years, due to diversified diets and over-nutrition, obesity problems continue to increase. Obesity actuates metabolic and chronic disorders, such as diabetes, hypertension and cardiovascular diseases, is harmful to human health and has serious consequences.

Obesity is a status in which excessive lipid accumulation in bodies enlarges adipocytes because the imbalance between intake and consumption of energy turns the excess energy into lipid accumulation in adipocytes or adipose tissues. Many known chemical synthetic drugs for weight loss have the effect of controlling obesity. When using chemical synthetic drugs for treating obesity, safety problems and side effects are uncertain factors to a human body. For instance, taking the diet pill Reductil for a long term causes dry mouth, insomnia, constipation, headache or other side effects, so that the patients' daily routines are affected, and they are reluctant to continue taking the medication. Moreover, adding chemical synthetic weight-loss drugs to foods in daily life for treating or preventing obesity may discomfort the patients on the diet due to large amounts of synthetic chemicals used in the weight-loss drugs. Conventional weight-loss drugs are accompanied by many side effects discomforting patients, and chemical synthetic substances entering a body may be harmful to the patients' health or cause many side effects, so these medicines are not recommended.

In addition, Antrodia camphorata, which is used in Taiwanese folk medicine, has multiple biological activities. Current research studies have shown Antrodia camphorata to have the functions of anti-tumor, anti-inflammatory/immunomodulatory, anti-virus, anti-allergy, anti-hypertension, inhibition of platelet aggregation, blood glucose level reduction, cholesterol level reduction, and hepatoprotective activities. However, the potential of Antrodia camphorata in the inhibition of adipocyte differentiation and anti-obesity property has not been studied and confirmed.

As shown above, the weight-loss drugs used currently have adverse effects on human bodies to a considerable extent, so how to choose natural weight-loss drugs, reduce side effects and achieve the goal of weight loss are the problems which the present invention aims to solve.

### SUMMARY OF THE INVENTION

A primary object of the present invention is to solve the problems of the side effects of chemical weight-loss drugs on a human body and to provide a method for reducing obesity by using Antrodia camphorata, which avoids the discomforts and side effects resulting from the chemical synthetic weight-loss drugs which are used in treating obesity.

To achieve the above object, the present invention provides a method by using Antrodia camphorata to reduce lipid accumulation in adipocytes, thereby improving symptoms of obesity.

In one aspect of the present invention, the composition of Antrodia camphorata comprises artificially cultured Antrodia camphorata, such as Antrodia camphorata cultured in/on artificial media.

In one aspect of the present invention, the composition of Antrodia camphorata comprises dried fruiting bodies of Antrodia camphorata.

In one aspect of the present invention, the composition of Antrodia camphorata is separated from a water-soluble extract of the dried fruiting bodies of Antrodia camphorata.

In one aspect of the present invention, the water-soluble extract of Antrodia camphorata comprises polysaccharide ingredients and non-polysaccharide ingredients, and the water-soluble extract of Antrodia camphorata is directly applied to inhibit at least one of the mRNA expressions of C/EBPα, C/EBPβ, PPARγ, FAS, and aP2 in the adipocytes.

In one aspect of the present invention, the water-soluble extract of Antrodia camphorata is further separated into polysaccharide and non-polysaccharide ingredients, and one of the polysaccharide ingredients and the non-polysaccharide ingredients is selected and used to inhibit at least one of mRNA expressions of C/EBPα, C/EBPβ, PPARγ, FAS, and aP2 in the adipocytes.

In one aspect of the present invention, the composition of Antrodia camphorata is used via an oral administration.

To achieve the above object, the present invention further provides a method for controlling obesity by using Antrodia camphorata, comprising steps of: (a) providing dried fruiting bodies of Antrodia camphorata; (b) extracting a composition of Antrodia camphorata from the dried fruiting bodies of Antrodia camphorata; and (c) administering the composition of Antrodia camphorata for reducing lipid accumulation in adipocytes and improving symptoms of obesity.

In one aspect of the present invention, the composition of Antrodia camphorata in the step (b) is a water-soluble extract of Antrodia camphorata comprising polysaccharide ingredients and non-polysaccharide ingredients.

In one aspect of the present invention, the water-soluble extract of Antrodia camphorata in the step (b) is further separated into the polysaccharide ingredients and the non-polysaccharide ingredients, and one of the polysaccharide ingredients and the non-polysaccharide ingredients is selected to proceed with the step (c).

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a preparation flowchart of water-soluble extracts, polysaccharide fractions and non-polysaccharide fractions of Antrodia camphorata.

FIG. 2 is a chromatography spectrum of polysaccharide fractions of Antrodia camphorata in the present invention.

FIG. 3 is a diagram illustrating differentiation stages at which 3T3-L1 cells are given and co-cultivated with the water-soluble extract of Antrodia camphorata (200 μg/mL).

FIG. 4 is a diagram illustrating the results of the inhibitive effects of water-soluble extracts of Antrodia camphorata on lipid accumulation in 3T3-L1 cells at different differentiation stages.

FIG. 5 is a diagram illustrating the results of the inhibitive effects of water-soluble extracts, polysaccharide fractions and non-polysaccharide fractions of Antrodia camphorata on lipid accumulation in 3T3-L1 cells at differentiation stages.

FIGS. 6A-6E are diagrams illustrating the results of the effects of water-soluble extracts, polysaccharide fractions and non-polysaccharide fractions of Antrodia camphorata on mRNA expressions of C/EBPβ, C/EBPα, PPARγ, FAS, and aP2 in 3T3-L1 cells.

FIG. 7 is a method flowchart of using Antrodia camphorata for controlling obesity.

### DETAILED DESCRIPTION OF THE PREFERRED EMBODIMENTS

By reference to the accompanying drawings, the specific embodiments of the methods for controlling obesity by using Antrodia camphorata are described in detail as follows:

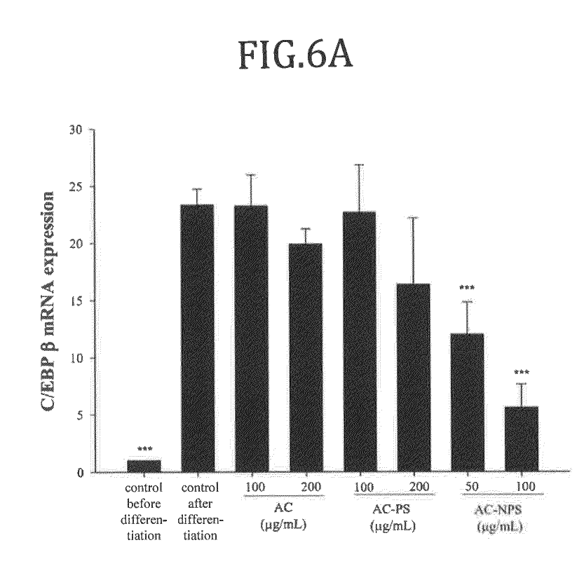

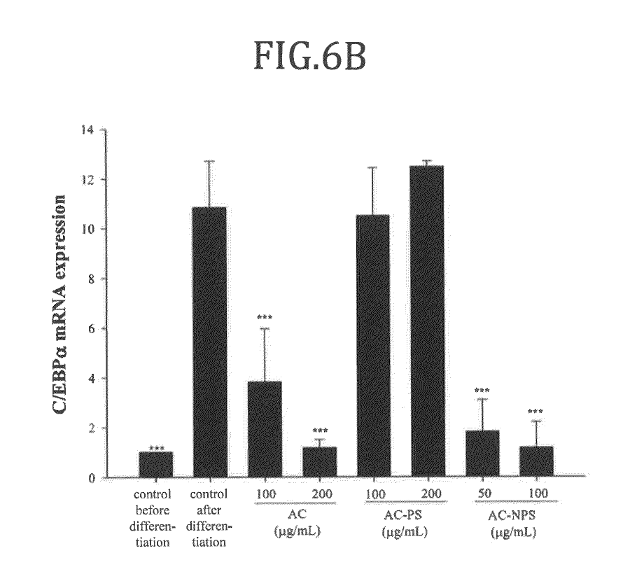

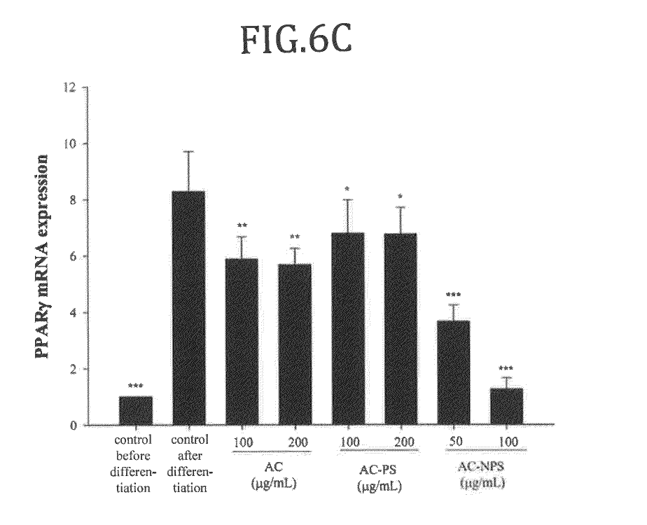

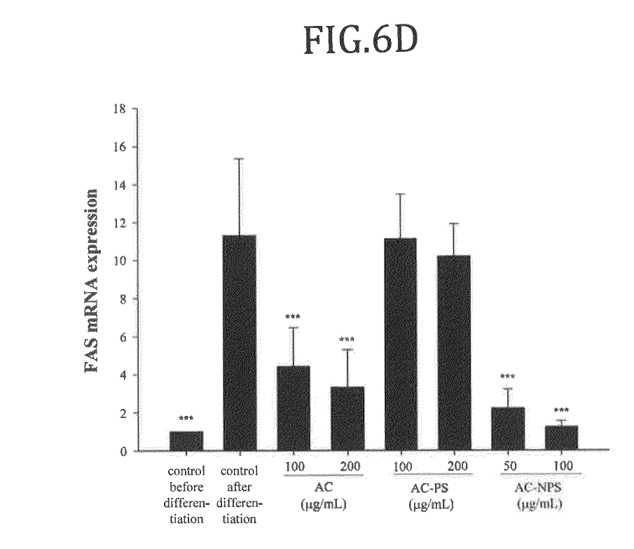

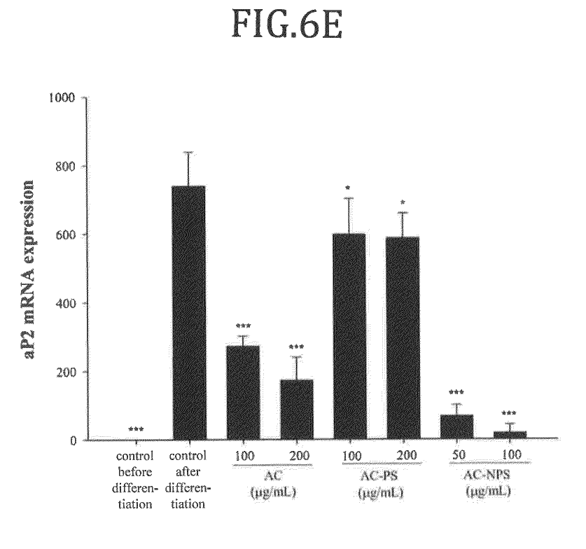

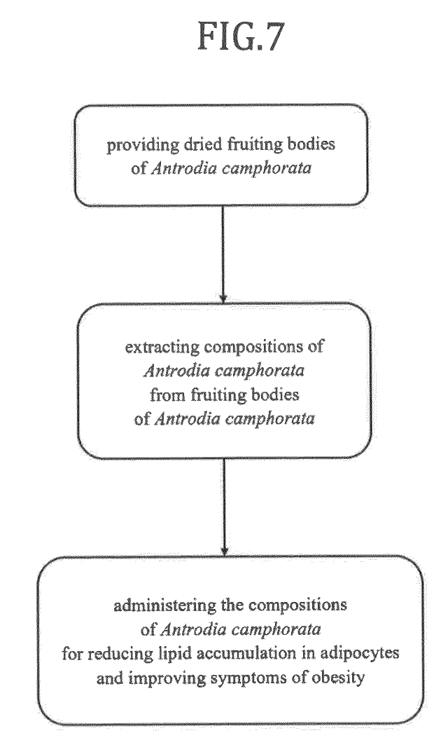

Referring to FIGS. 6A-6C and FIG. 7, one embodiment of the present invention provides a method for controlling obesity by using Antrodia camphorata, which applies Antrodia camphorata to reduce lipid accumulation in adipocytes to improve the symptoms of obesity. The source of the compositions of Antrodia camphorata used is derived from artificially cultured Antrodia camphorata cultured in/on artificial media. The methods of the present invention are described as follows.

### Preparation of Water-Soluble Extracts of Antrodia camphorata

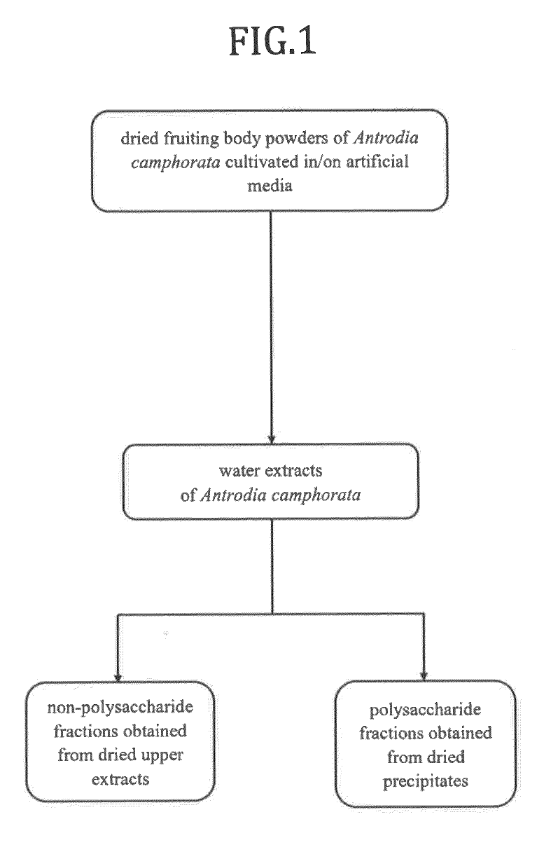

In the embodiment of the present invention, the source of the compositions of Antrodia camphorata used is the dried fruiting bodies of Antrodia camphorata. Antrodia camphorata cultivated on artificial nutrient media is used as the material. The compositions of Antrodia camphorata are separated from water-soluble extracts of the Antrodia camphorata fruiting bodies, and the preparation method of the water-soluble extracts is as shown in FIG. 1. The water-soluble extracts of Antrodia camphorata contain polysaccharide and non-polysaccharide ingredients. The preparation method is that after the dried fruiting bodies of Antrodia camphorata are grounded into powders, the powders are extracted continuously for 6 hours in hot water at 80° C. in the ratio of 1:100 (w/w). First the extracts are filtered through a Buchner funnel, and the remaining powders are added to the same volume of hot water at 80° C. for extraction for 6 hours, and water-soluble extracts of the Antrodia camphorata are obtained. After filtered, the water-soluble extracts are added into 95% ethanol which is three times the volume of the water-soluble extracts at 4° C. overnight, and are centrifuged at 9000×g for 20 minutes next day. The precipitates of Antrodia camphorata obtained through the centrifuge process are dissolved in distilled and deionized water (DD water) at 65° C., and subsequently freeze-dried to produce the crude polysaccharide extracts of Antrodia camphorata. The water-soluble extracts of Antrodia camphorata dissolved in ethanol are concentrated under low pressure and freeze-dried to produce the crude non-polysaccharide extracts of Antrodia camphorata. The crude polysaccharide and non-polysaccharide extracts of Antrodia camphorata obtained through the freeze-drying process are re-dissolved in dimethyl sulfoxide (DMSO) to produce polysaccharide fractions of Antrodia camphorata (AC-PS) and non-polysaccharide fractions of Antrodia camphorata (AC-NPS) for proceeding to the subsequent experiments.

### Analysis of Water-Soluble Extract Compositions of Antrodia camphorata

The polysaccharides of Antrodia camphorata contained in the polysaccharide fractions of Antrodia camphorata are analyzed. A solution of polysaccharide fractions of Antrodia camphorata (AC-PS) in milli-Q water was diluted to give a concentration of 1 mg/ml and was then filtered through a 0.22-μm filter (Millipore, Billerica, Mass., USA) before injection into the size-exclusion chromatography (SEC) column. The flow rate was 0.5 mL/min, with deionized water as the eluent. A calibration curve was constructed using an authentic standard, Sodex P-82 series (Showa Denko America, Mentor, Ohio, USA) containing polymaltotriose with molecular weights of 78.8×10⁴, 40.4×10⁴, 21.2×10⁴, 4.73×10⁴, and 1.18×10⁴ Da. The TriSec software program was used to acquire and analyze the Viscotek data. SEC signal detection was performed using a ViscoTek model TDA-3-1 relative viscometer (ViscoTek, Houston, Tex., USA).

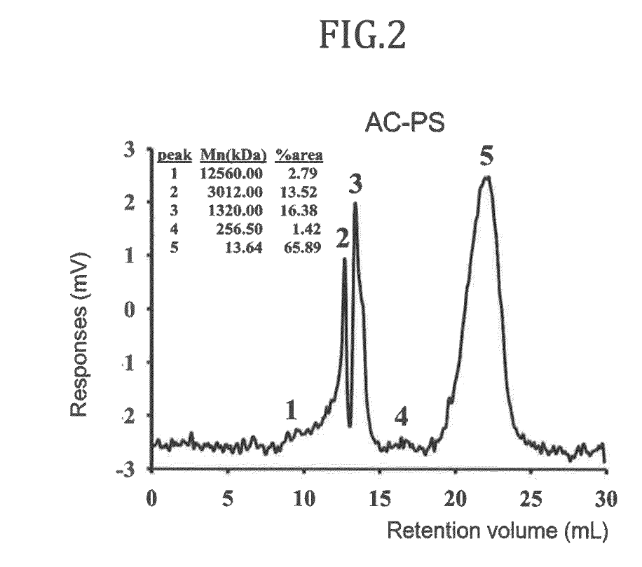

As shown in FIG. 2, the average molecular weight (Mn), the maximal molecular weight (Mw) and the proportion (% area) of polysaccharides in the polysaccharide fraction of Antrodia camphorata are obtained by analyzing the signal strength peaks.

The results have shown that the signal distribution of the polysaccharide fractions of Antrodia camphorata (AC-PS) is mainly divided into five areas. The first area (peak 1) has an average molecular weight of 12560 kDa, the second area (peak 2) has an average molecular weight of 3012 kDa, the third area (peak 3) has an average molecular weight of 1320 kDa, the fourth area (peak 4) has an average molecular weight value 256.5 kDa, and the fifth area (peak 5) has average molecular weight of 13.64 kDa. According to the distribution result of each area, the fifth area (peak 5) has the highest proportion of 65.89%, followed by the third area (peak 3) and the second area (peak 2), proportions of which are 16.38% and 13.52% respectively. The first area (peak 1) has a proportion of 2.79% while the forth area (peak 4) has the lowest proportion of 1.42%. Therefore, the molecular weight less than 14 kDa are the main compositions in the polysaccharide fractions of Antrodia camphorata.

The polysaccharide fractions of Antrodia camphorata are hydrolyzed in 4.95 N trifluoroacetic acid (TFA) in a water bath at 80° C. for 24 hours. The hydrolyzed polysaccharide fractions of Antrodia camphorata are analyzed via a high-performance anion-exchange chromatography (HPAEC). The contents of mono-saccharides in the specimen are analyzed and compared with standard samples, including myoinositol (99%), sorbitol (98%), fucose (99%), galactose (99%), glucose (99.5%), and mannose (99%).

In the contents of mono-saccharides in the polysaccharides fraction of Antrodia camphorata, galactose is the most, containing approximately 327.68±0.64 μmol per gram of polysaccharides, followed by fucose, containing about 46.24±0.81 μmol per gram of polysaccharides. In addition, carbohydrates, such as glucose, sorbitol, myoinositol, mannose, fructose, galactosamine and glucosamine, are also measured, but the levels thereof are low.

### Effects of Water-Soluble Extracts of Antrodia camphorata on Lipid Accumulation in 3T3-L1 Cells at Different Differentiation Stages

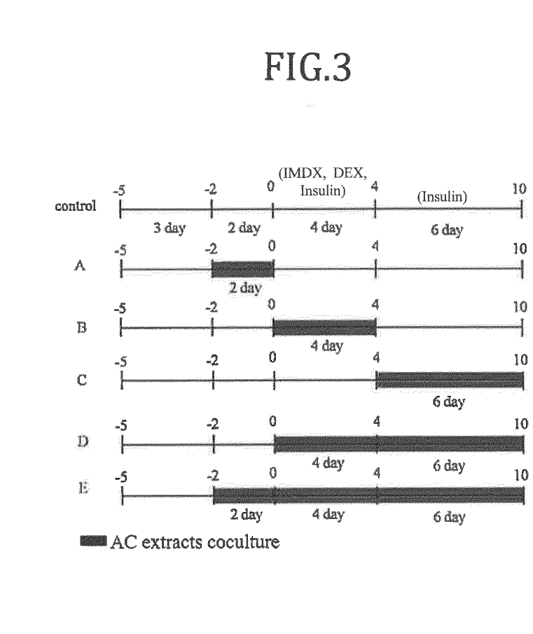

By using 3T3-L1 cells as the material for the study, the effects of the water-soluble extracts of Antrodia camphorata on the lipid accumulation in the 3T3-L1 cells at different differentiation stages are analyzed after the 3T3-L1 cells are stimulated by differentiation agents. As shown in FIG. 3, the 3T3-L1 cells are given and co-cultivated with the water-soluble extracts of Antrodia camphorata (200 μg/mL) at the different differentiation stages. Oil red O staining for lipid droplet is conducted on the 3T3-L1 cells on the tenth day of differentiation, and the inhibition ratios of the lipid accumulation are observed. In the control group, no water-soluble extract of Antrodia camphorata is added into the 3T3-L1 cells. In group A, the water-soluble extracts of Antrodia camphorata are added into the 3T3-L1 cells only from two days before the differentiation (day-2) to the beginning of the differentiation (day 0). In group B, the water-soluble extracts of Antrodia camphorata are added into the 3T3-L1 cells from the beginning of the differentiation (day 0) to four days after the differentiation. In C group, the water-soluble extracts of Antrodia camphorata are added into the 3T3-L1 cells from four days after the differentiation to ten days after the differentiation. In group D, the water-soluble extracts of Antrodia camphorata are added into the 3T3-L1 cells from the beginning of the differentiation (day 0) to ten days after the differentiation. In E group, the water-soluble extracts of Antrodia camphorata are added into the 3T3-L1 cells from two days before the differentiation (day-2) to ten days after the differentiation.

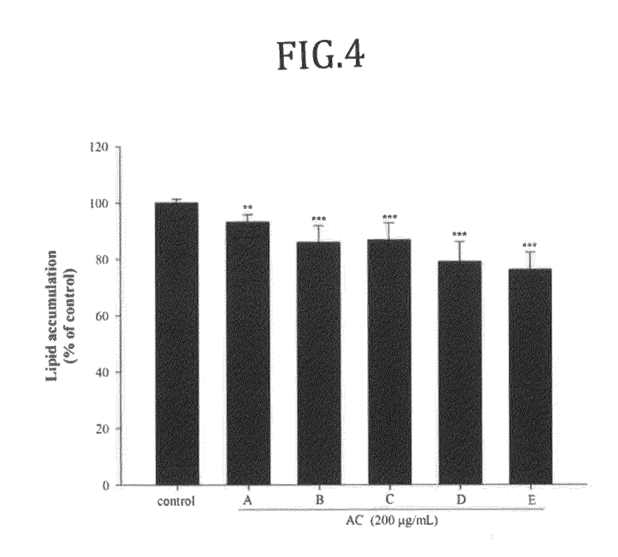

The results are observed and obtained after the water-soluble extracts of Antrodia camphorata are given and co-cultivated at the different differentiation stages. As shown in FIG. 4, there are inhibitive effects on lipid accumulation when the water-soluble extracts are added prior to (in group A) or during (in group B, C, D, and E) the differentiation. When the control group is regarded as 100% (no lipid accumulation inhibition), the effectiveness of the lipid accumulation is reduced by 6.8% 14%, 13.3%, 20.9%, 23.7% respectively in order of group A, B, C, D and E. The inhibitive effects on lipid accumulation also vary when the water-soluble extracts of Antrodia camphorata are added in the 3T3-L1 cells at the differentiation stages cells. In group D and group E, the inhibitive effects are the most significant. The subsequent experiments of the water-soluble extracts and their fractions (the polysaccharide fractions and non-polysaccharide fractions) of Antrodia camphorata are conducted in the method of group D for explaining the action and mechanism of the inhibition of the lipid accumulation.

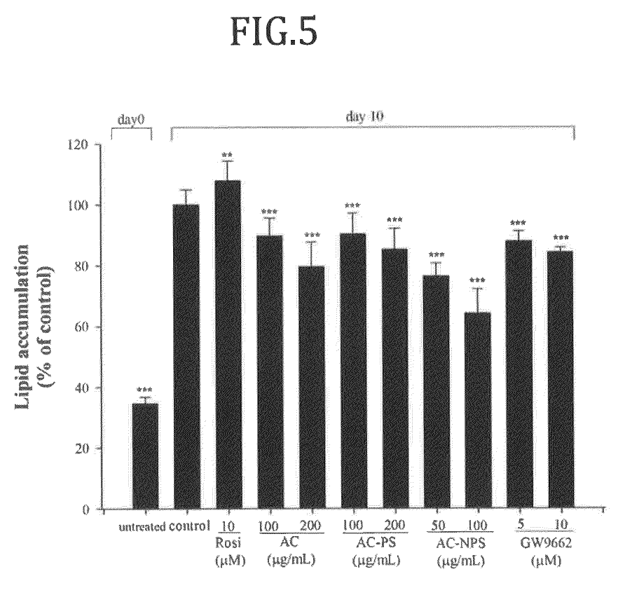

Referring to FIG. 5, when the water-soluble extracts, the polysaccharide fractions, and the non-polysaccharide fractions of Antrodia camphorata are added at the differentiation stages, they all have significant effects on the inhibition of lipid accumulation in the 3T3-L1 cells. The percentages of the lipid accumulation are respectively reduced by 10.3% and 20.4% under concentrations of 100 μg/mL and 200 μg/mL of the water-soluble extracts of Antrodia camphorata. The percentages of the lipid accumulation are respectively reduced by 9.8% and 14.8% under concentrations of 100 μg/mL and 200 μg/mL of the polysaccharide fractions of Antrodia camphorata. The percentages of the lipid accumulation are respectively reduced by 23.7% and 35.8% under concentrations of 50 μg/mL and 100 μg/mL of the non-polysaccharide fractions of Antrodia camphorata. Rosiglitazone in FIG. 5, abbreviated as Rosi, is the agonist of PPARγ (a lipogenic regulatory gene) and increases lipid accumulation by activating PPARγ to induce lipogenesis. GW9662 is the antagonist of PPARγ and decrease lipid accumulation by inhibiting the expression of PPARγ. The results show that the non-polysaccharide fractions of Antrodia camphorata reduce lipid accumulation most effectively, the inhibition effectiveness increases with the increasing concentrations, and the sizes and accumulation conditions of the lipid droplets decrease significantly with the increasing concentrations, wherein the non-polysaccharide fractions of Antrodia camphorata (AC-NPS) show the most significant effect.

### Effects of Water-Soluble Extracts of Antrodia camphorata and Fractions Thereof (Polysaccharide Fractions of Antrodia camphorata and Non-Polysaccharide Fractions of Antrodia camphorata) on the mRNA Expressions of Genes Related to Differentiation and Lipid Metabolism in 3T3-L1 Cells

C/EBPβ, C/EBPα, PPARγ, FAS and aP2 all play important roles in the adipocyte differentiation process. The water-soluble extracts of Antrodia camphorata, the polysaccharide fractions of Antrodia camphorata, and the non-polysaccharide fractions of Antrodia camphorata are directly applied to inhibiting at least one of the mRNA expressions of C/EBPβ, C/EBPα, PPARγ, aP2 and FAS in adipocytes, wherein the mRNA sequences of C/EBPβ, C/EBPα, PPARγ, aP2 and FAS are called SEQ ID NO: 1, 2, 3, 4 and 5, respectively, and listed in the following sequence listing. The water-soluble extracts of Antrodia camphorata, the polysaccharide fractions of Antrodia camphorata, the non-polysaccharide fractions of Antrodia camphorata are added in the differentiation period, and the mRNA expressions of C/EBPβ, C/EBPα, PPARγ, FAS and aP2 in cells are measured with real time-PCR on the fourth day of the differentiation. The results are shown in FIGS. 6A-6E. The mRNA expressions of C/EBPα, PPARγ, FAS, and aP2 are inhibited significantly at concentrations of 100 μg/ mL or 200 μg/mL of the water-soluble extracts of Antrodia camphorata. When the control group is regarded as 100% (no lipid accumulation inhibition), the percentages of the gene expressions are calculated under concentrations of 100 μg/mL and 200 μg/mL of the water-soluble extracts of Antrodia camphorata. The C/EBP α expression levels are respectively reduced 65% and 89%, the PPARγ expression levels are respectively reduced by 29% and 31%, the FAS expression levels are respectively reduced by 61% and 71%, and the aP2 expression levels are reduced 63% and 77%.

The PPARγ and aP2 mRNA expressions are significantly inhibited under concentrations of 100 μg/mL and 200 μg/mL of the polysaccharide fractions of Antrodia camphorata. When the control group is regarded as 100% (no lipid accumulation inhibition), the PPARγ expression levels are both reduced by 18%, and the aP2 expression levels are respectively reduced by 22% and 21% under concentrations of 100 μg/mL and 200 μg/mL of the polysaccharide fractions of Antrodia camphorata.

The C/EBPβ, C/EBPα, PPARγ, FAS, and aP2 mRNA expressions are significantly inhibited under concentrations of 50 μg/mL and 100 μg/mL of the non-polysaccharide fractions of Antrodia camphorata. When the control group is regarded as 100% (no lipid accumulation inhibition), the C/EBPβ expression levels are respectively reduced by 49% and 76%, the C/EBPα expression levels are respectively reduced by 83% and 89%, the PPARγ expression levels are respectively reduced by 56% and 85%, the FAS expression levels are respectively reduced by 80% and 89%, and the aP2 expression levels are respectively reduced by 91% and 98% under concentrations of 50 μg/mL and 100 μg/mL of the non-polysaccharide fractions of Antrodia camphorata.

Referring to FIG. 7, the present invention provides a method used for controlling obesity by using Antrodia camphorata, comprising following steps: (a) providing dried fruiting bodies of Antrodia camphorata; (b) extracting compositions of Antrodia camphorata from fruiting bodies of Antrodia camphorata; and (c) administering the compositions of Antrodia camphorata for reducing lipid accumulation in adipocytes and improving the symptoms of obesity.

As the method described above, the source of the compositions of Antrodia camphorata used is derived from Antrodia camphorata cultivated on artificial media, and the compositions of Antrodia camphorata in the step (b) are water-soluble extracts of Antrodia camphorata comprising polysaccharide ingredients and non-polysaccharide ingredients.

As the method described above, the water-soluble extracts of Antrodia camphorata are further separated into the polysaccharide ingredients and the non-polysaccharide ingredients, and one of the polysaccharide ingredients and the non-polysaccharide ingredients is selected to proceed to the step (c).

In the step (c), the water-soluble extracts of Antrodia camphorata containing the polysaccharide ingredients and the non-polysaccharide ingredients are applied directly to inhibiting at least one of mRNA expressions of C/EBPα, C/EBPβ, PPARγ, FAS and aP2 in adipocytes; and alternatively, one of the polysaccharide fractions of Antrodia camphorata and the non-polysaccharide fractions of Antrodia camphorata can be selected and used to inhibit at least one of mRNA expressions of C/EBPα, C/EBPβ, PPARγ, FAS, and aP2 in adipocytes for reducing lipid accumulation.

In summary, the water-soluble extracts of Antrodia camphorata, the polysaccharide fractions of Antrodia camphorata, and the non-polysaccharide fractions of Antrodia camphorata are prepared by using Antrodia camphorata cultivated on artificial nutrient media as the raw material, and the biological activities are analyzed by the experimental cell models in the present invention. In the results of the cell experiments, it has been found that the effectiveness of the lipid accumulation in the 3T3-L1 cells is significantly reduced when the 3T3-L1 cells are given and co-cultivated with the water-soluble extracts of Antrodia camphorata at the differentiation stages. Moreover, the polysaccharide fraction of Antrodia camphorata, and the non-polysaccharide fraction of Antrodia camphorata are prepared, and the results show that the non-polysaccharide fractions of Antrodia camphorata have more significant effects on the inhibition of lipid accumulation. The water-soluble extracts of Antrodia camphorata, the polysaccharide fractions of Antrodia camphorata, and the non-polysaccharide fractions of Antrodia camphorata all can inhibit the expressions of several genes responsible for adipocyte differentiation and lipogenesis. The method for controlling obesity by using Antrodia camphorata in the present invention does have the effect of alleviating obesity.

The present invention has been described with a preferred embodiment thereof and it is understood that various modifications, without departing from the spirit of the present invention, are in accordance with the embodiments of the present invention. Hence, the embodiments described are intended to cover the modifications within the scope and the spirit of the present invention, rather than to limit the present invention.

1. A method for controlling obesity by using Antrodia camphorata, comprising: applying a composition of Antrodia camphorata to reduce lipid accumulation in adipocytes to improve symptoms of obesity, wherein a water-soluble extract is separated from Antrodia camphorate, a polysaccharide extract and a non-polysaccharide extract are separated from the water-soluble extract, and the composition selected from the non-polysaccharide extract is used to reduce lipid accumulation in adipocytes.

2. The method as claimed in claim 1, wherein the composition of Antrodia camphorata comprises artificially cultured Antrodia camphorata.

3. The method as claimed in claim 2, wherein the composition of Antrodia camphorata comprises dried fruiting bodies of Antrodia camphorata.

4. (canceled)

5. The method as claimed in claim 1, wherein the non-polysaccharide extract is directly applied to inhibit at least one type of the mRNA expressions of CCAAT-enhancer-binding protein α (C/EBPα), CCAAT-enhancer-binding protein β (C/EBPβ), peroxisome proliferator-activated receptors γ (PPARγ), fatty acid synthase (FAS), and adipocyte protein 2 (aP2).

6. (canceled)

7. The method as claimed in claim 1, wherein the composition of Antrodia camphorata is used via an oral administration.

8. A method for controlling obesity by using Antrodia camphorata, comprising steps of:
(a) providing dried fruiting bodies of Antrodia camphorata;
(b) extracting a composition of Antrodia camphorata from the dried fruiting bodies of Antrodia camphorata; and
(c) separating a water-soluble extract from Antrodia camphorate;
(d) separating a polysaccharide extract and a non-polysaccharide extract from the water-soluble extract;
(e) administering the non-polysaccharide extract for reducing lipid accumulation in adipocytes and improving symptoms of obesity.

9. (canceled)

10. (canceled)
